(19) United States
(12) Patent Application Publication
(10) Pub. No.: US 2021/0023072 A1
(43) Pub. Date: Jan. 28, 2021

(54) THERAPEUTIC USES OF GLP1R AGONISTS

(71) Applicant: vTv Therapeutics LLC, High Point, NC (US)

(72) Inventors: Jennifer L.R. Freeman, Winston-Salem, NC (US); Maria Carmen Valcarce Lopez, Oak Ridge, NC (US)

(21) Appl. No.: 17/069,453

(22) Filed: Oct. 13, 2020

Related U.S. Application Data
(63) Continuation of application No. PCT/US2019/030110, filed on May 1, 2019.
(60) Provisional application No. 62/668,384, filed on May 8, 2018.

(51) Int. Cl. A61K 31/4741; A61P 3/10; A61K 9/00

(52) U.S. Cl. CPC A61K 31/4741; A61K 9/0053; A61P 3/10

(57) ABSTRACT

Methods of using glucagon-like peptide 1 receptor (GLP1R) agonists are generally disclosed herein. In certain aspects, the disclosure provides methods of treating type 2 diabetes that include administering a GLP1R agonist according to certain dosage regimens. In certain other aspects, the disclosure provides methods of treating obesity that include administering a GLP1R agonist according to certain dosage regimens. In certain other aspects, the disclosure provides methods of lowering glycated hemoglobin (for example, lowering HbA1c) that include administering a GLP1R agonist according to certain dosage regimens. Compositions containing GLP1R agonists and their manufacture, for example, for use as a medicament are also disclosed herein.

### TECHNICAL FIELD

Methods of using glucagon-like peptide 1 receptor (GLP1R) agonists are generally disclosed herein. In certain aspects, the disclosure provides methods of treating type 2 diabetes that include administering a GLP1R agonist according to certain dosage regimens. In certain other aspects, the disclosure provides methods of treating obesity that include administering a GLP1R agonist according to certain dosage regimens. In certain other aspects, the disclosure provides methods of lowering glycated hemoglobin (for example, lowering HbA1c) that include administering a GLP1R agonist according to certain dosage regimens. Compositions containing GLP1R agonists and their manufacture, for example, for use as a medicament are also disclosed herein.

### BACKGROUND

Diabetes mellitus type 2 (type 2 diabetes) is a chronic metabolic disorder characterized by a number of symptoms, including, but not limited to, elevated blood-glucose levels, insulin resistance, impaired insulin secretion, and hyperglycemia. Symptoms associated with type 2 diabetes tend to manifest themselves gradually and progressively, becoming worse and greater in number as the disease progresses. If not treated well, type 2 diabetes eventually can lead to heart disease, stroke, blindness (due to diabetic retinopathy), kidney failure, and poor blood circulation to the limbs (which can result in the need to amputate limbs, such as feet and toes, that no longer benefit from sufficient circulation). Type 2 diabetes and its related disorders, such as obesity, pose a major public health problem throughout the world.

The causes of type 2 diabetes are multifactorial in nature. But obesity, combined with insufficient physical activity, is the leading contributing factor. Genetic factors can also increase the likelihood that one develops type 2 diabetes. Treatment regimens vary. In many cases, type 2 diabetes may be managed by maintaining a normal weight, exercising regularly, and eating properly. But such measures are often insufficient, as patients may resist compliance because such measures involve lifestyle changes. Therefore, antidiabetic medications, such as metformin, are often prescribed. But metformin therapy often fails to affect disease progression in a clinically meaningful way.

Various second-line antidiabetic medications are also used. Glucagon-like peptide 1 (GLP1) analogs and glucagon-like peptide 1 receptor (GLP1R) agonists are a class of therapies that have shown particular promise in treating diabetes. Non-peptide GLP1R agonists have also been discovered, such as those disclosed in U.S. Pat. Nos. 7,727,983 and 8,383,644. The protein-based therapies are generally delivered by intravenous injection, which causes a certain degree of inconvenience and discomfort for the patient. Some protein-based therapies are being developed for oral administration. In some instances, oral administration may be a more desirable alternative. And while some compounds in this class may be amenable to oral administration, effective regimens for oral delivery are still under development.

Thus, there is a continuing need to develop effective dosing regimens for the oral delivery of GLP1R agonists.

### SUMMARY

The present disclosure generally provides methods of treating type 2 diabetes and related conditions, such as elevated glycated hemoglobin levels, obesity, and lack of glycemic control. It was surprisingly discovered that certain GLP1R agonists exhibit non-linear dose dependent activity when dosed in vivo, where after reaching a maximum efficacy point, increased doses show decreasing efficacy. Therefore, it was discovered that one could improve the efficacy of the compounds, in certain respects, by using lower doses than expected. This also had the concomitant benefit of reducing the likelihood of side-effects in certain subjects.

In a first aspect, the disclosure provides methods of lowering glycated hemoglobin levels in a subject, the methods comprising administering to a subject in need thereof from 0.1 to 5.0 mg/kg or between 10 mg and 500 mg daily of a glucagon-like peptide 1 receptor (GLP1R) agonist. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In a second aspect, the disclosure provides methods of treating type 2 diabetes, the methods comprising administering to a subject in need thereof from 0.1 to 5.0 mg/kg or between 10 mg and 500 mg daily of a glucagon-like peptide 1 receptor (GLP1R) agonist. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In a third aspect, the disclosure provides methods of reducing body weight, the methods comprising administering to a subject in need thereof from 0.1 to 5.0 mg/kg or between 10 mg and 500 mg daily of a glucagon-like peptide 1 receptor (GLP1R) agonist. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In a fourth aspect, the disclosure provides methods of treating obesity, the methods comprising administering to a subject in need thereof from 0.1 to 5.0 mg/kg or between 10 mg and 500 mg daily of a glucagon-like peptide 1 receptor (GLP1R) agonist. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In a fifth aspect, the disclosure provides methods of improving glycemic control, the methods comprising administering to a subject in need thereof from 0.1 to 5.0 mg/kg or between 10 mg and 500 mg daily of a glucagon-like peptide 1 receptor (GLP1R) agonist. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In a sixth aspect, the disclosure provides glucagon-like peptide 1 receptor (GLP1R) agonists for use in lowering elevated glycated hemoglobin levels in a subject, wherein the GLP1R agonist is administered to a subject in an amount from 0.1 to 5.0 mg/kg or between 10 mg and 500 mg daily. In some embodiments, the GLP1R agonist is (S)-2-{[(3S,8S)-3-[4-(3,4-dichloro-benzyloxy)-phenyl]-7-((a)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In a seventh aspect, the disclosure provides glucagon-like peptide 1 receptor (GLP1R) agonists for use in treating type 2 diabetes, wherein the GLP1R agonist is administered to a subject in an amount from 0.1 to 5.0 mg/kg or between 10 mg and 500 mg daily. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In an eighth aspect, the disclosure provides glucagon-like peptide 1 receptor (GLP1R) agonists for use in treating obesity, wherein the GLP1R agonist is administered to a subject in an amount from 0.1 to 5.0 mg/kg or between 10 mg and 500 mg daily. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In a ninth aspect, the disclosure provides glucagon-like peptide 1 receptor (GLP1R) agonists for use in lowering body weight, wherein the GLP1R agonist is administered to a subject in an amount from 0.1 to 5.0 mg/kg or between 10 mg and 500 mg daily. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In a tenth aspect, the disclosure provides glucagon-like peptide 1 receptor (GLP1R) agonists for use in improving glycemic control, wherein the GLP1R agonist is administered to a subject in an amount from 0.1 to 5.0 mg/kg or between 10 mg and 500 mg daily. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In an eleventh aspect, the disclosure provides uses of glucagon-like peptide 1 receptor (GLP1R) agonists in the manufacture of a medicament for lowering elevated levels of glycated hemoglobin in a subject, wherein the medicament is prepared to be administered to a subject in an amount from 0.1 to 5.0 mg/kg or between 10 mg and 500 mg daily. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In an twelfth aspect, the disclosure provides uses of glucagon-like peptide 1 receptor (GLP1R) agonists in the manufacture of a medicament for treating type 2 diabetes, wherein the medicament is prepared to be administered to a subject in an amount from 0.1 to 5.0 mg/kg or between 10 mg and 500 mg daily. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In an thirteenth aspect, the disclosure provides uses of glucagon-like peptide 1 receptor (GLP1R) agonists in the manufacture of a medicament for treating obesity, wherein the medicament is prepared to be administered to a subject in an amount from 0.1 to 5.0 mg/kg or between 10 mg and 500 mg daily. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In an fourteenth aspect, the disclosure provides uses of glucagon-like peptide 1 receptor (GLP1R) agonists in the manufacture of a medicament for lowering elevated body weight, wherein the medicament is prepared to be administered to a subject in an amount from 0.1 to 5.0 mg/kg or between 10 mg and 500 mg daily. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

In an fifteenth aspect, the disclosure provides uses of glucagon-like peptide 1 receptor (GLP1R) agonists in the manufacture of a medicament for improving glycemic control, wherein the medicament is prepared to be administered to a subject in an amount from 0.1 to 5.0 mg/kg or between 10 mg and 500 mg daily. In some embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some other embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In one embodiment, the GLP1R agonist is administered orally.

Other aspects and embodiments are set forth in the foregoing drawings, detailed description, and claims.

### BRIEF DESCRIPTION OF THE DRAWINGS

FIG. 1. Data points represent the change from baseline at Week 12 by dose (mg/kg) for HbA1c (%) for completers by treatment arm (placebo=X; 150 mg once daily (QPM)=•).

FIG. 2. Data points represent the change from baseline at Week 12 by dose (mg/kg) for HbA1c (%) for completers by treatment arm (placebo=X; 150 mg twice daily (BID)=•).

FIG. 3. Data points represent change from baseline at Week 12 by dose (mg/kg) for fasting plasma glucose (mg/dL) for completers by treatment arm (placebo=X; 150 mg once daily (QPM)=•).

FIG. 4. Data points represent change from baseline at Week 12 by dose (mg/kg) for fasting plasma glucose (mg/dL) for completers by treatment arm (placebo=X; 150 mg twice daily (BID)=•).

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “administer” or “administering” means to introduce, such as to introduce to a subject a compound or composition. The term is not limited to any specific mode of delivery, and can include, for example, subcutaneous delivery, intravenous delivery, intramuscular delivery, intracisternal delivery, delivery by infusion techniques, transdermal delivery, oral delivery, nasal delivery, and rectal delivery. Furthermore, depending on the mode of delivery, the administering can be carried out by various individuals, including, for example, a health-care professional (e.g., physician, nurse, etc.), a pharmacist, or the subject (e.g., self-administration).

As used herein, “treat” or “treating” or “treatment” can refer to one or more of: delaying the progress of a disease, disorder, or condition; controlling a disease, disorder, or condition; ameliorating one or more symptoms characteristic of a disease, disorder, or condition; or delaying the recurrence of a disease, disorder, or condition, or characteristic symptoms thereof, depending on the nature of the disease, disorder, or condition and its characteristic symptoms.

As used herein, “subject” refers to any mammal such as, but not limited to, humans, horses, cows, sheep, pigs, mice, rats, dogs, cats, and primates such as chimpanzees, gorillas, and rhesus monkeys. In some embodiments, the “subject” is a human. In some such embodiments, the “subject” is a human who exhibits one or more symptoms characteristic of a disease, disorder, or condition. The term “subject” does not require one to have any particular status with respect to a hospital, clinic, or research facility (e.g., as an admitted patient, a study participant, or the like).

As used herein, the term “pharmaceutical composition” is used to denote a composition that may be administered to a mammalian host, e.g., orally, topically, parenterally, by inhalation spray, or rectally, in unit dosage formulations containing conventional non-toxic carriers, diluents, adjuvants, vehicles and the like. The term “parenteral” as used herein, includes subcutaneous injections, intravenous, intramuscular, intracisternal injection, or by infusion techniques.

As used herein, the term “pharmaceutically acceptable salt” refers to a salt of a compound which are generally prepared by reacting the free base with a suitable organic or inorganic acid or by reacting the acid with a suitable organic or inorganic base. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium, and valerate. When an acidic substituent is present, such as —COOH, there can be formed the ammonium, morpholinium, sodium, potassium, barium, calcium salt, and the like, for use as the dosage form. When a basic group is present, such as amino or a basic heteroaryl radical, such as pyridyl, there can be formed an acidic salt, such as hydrochloride, hydrobromide, phosphate, sulfate, trifluoroacetate, trichloroacetate, acetate, oxalate, maleate, pyruvate, malonate, succinate, citrate, tartarate, fumarate, mandelate, benzoate, cinnamate, methanesulfonate, ethanesulfonate, picrate, and the like. In certain embodiments, the GLP1R agonist is a hydrochloride acid salt. In other embodiments, the GLP1R agonist is a tris(hydroxymethyl)aminomethane salt.

As used herein, the term “mass-equivalent,” when used in reference to a pharmaceutically acceptable salt of a compound, refers to the equivalent mass of the salt form of the compound needed to provide the same molar quantity of the compound. For example, the phrase “100 mg of 3-(dimethylamino)propionic acid, or the mass-equivalent of the hydrochloride salt thereof” refers, in the second part of the phrase, to an amount of 3-(dimethylamino)propionic acid hydrochloride needed to provide the same molar quantity of 3-(dimethylamino)propionic acid as 100 mg of 3-(dimethylamino)propionic acid. In this case, 3-(dimethylamino)propionic acid has a molecular weight of 117.15 g/mol and 3-(dimethylamino)propionic acid hydrochloride has a molecular weight of 153.61 g/mol. Thus, the mass-equivalent amount of 3-(dimethylamino)propionic acid hydrochloride to 100 mg of 3-(dimethylamino)propionic acid is 131.12 mg. The same analysis applies when using units such as mg/kg.

As used herein, the unit term “mg/kg” refers to the mass (measured in mg) of compound administered to a subject per the mass (measured in kg) of the subject. For example, “administering 1.0 mg/kg daily to a subject” refers to administering 170 mg daily to a subject having a mass of 170 kg.

As used herein, “mix” or “mixed” or “mixture” refers broadly to any combining of two or more compositions. The two or more compositions need not have the same physical state; thus, solids can be “mixed” with liquids, e.g., to form a slurry, suspension, or solution. Further, these terms do not require any degree of homogeneity or uniformity of composition. This, such “mixtures” can be homogeneous or heterogeneous, or can be uniform or non-uniform. Further, the terms do not require the use of any particular equipment to carry out the mixing, such as an industrial mixer.

As used herein, “optionally” means that the subsequently described event(s) may or may not occur. In some embodiments, the optional event does not occur. In some other embodiments, the optional event does occur one or more times.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, the term “glucagon-like peptide 1 receptor agonist” or “GLP1R agonist” is a compound that, at a given in vivo or in vitro concentration, functions as an agonist or partial agonist of the glucagon-like peptide 1 receptor, notwithstanding that the compound may exhibit some secondary (weaker) antagonism of the glucagon-like peptide 1 receptor at certain other concentrations. In some cases, the GLP1R agonists or agonists can be referred to as being “protein-based” or as being “non-protein.” As used herein in this context, the term “peptide-based” refers to a compound that contains one or more chains of six or more alpha-amino acids connected by amide linkages, and wherein the one or more chains of amino acids make up at least 40% by mass of the compound's mass. As used herein in this context, the term “non-peptide” or “non-protein” refers to a compound in which no more than 40% of its mass is made up by one or more chains of six or more alpha-amino acids connected by amide linkages. In some embodiments, the non-protein GLP1R agonists have a molecular weight of no more than 2000 Da, or a molecular weight of no more than 1500 Da, or a molecular weight of no more than 1200 Da.

Other terms are defined in other portions of this description, even though not included in this subsection.

### Dosage Regimens for GLP1R Agonists

In one or more of the aforementioned aspects, the disclosure provides methods of administering non-protein GLP1R agonists of GLP1R agonists to subjects in need thereof. In general, such methods include administering to a subject in need thereof from 0.1 to 3.0 mg/kg or between 10 mg and 500 mg daily of a GLP1R agonist. In some aspects and embodiments, the GLP1R agonist is a GLP1R agonist.

Any suitable GLP1R agonist or agonist can be used. Suitable non-limiting examples include compounds recited in U.S. Pat. No. 7,727,983 (such as example 86) and U.S. Pat. No. 8,383,644 (such as example 179).

In some embodiments of any of the aforementioned embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing. In some further such embodiments, the GLP1R agonist is (5)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid. In some other such embodiments, the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2). In some other such embodiments, the GLP1R agonist is a combination of any of the above.

In some embodiments of any of the aforementioned embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing. In some such embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-proionic acid. In some other such embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1). In some other such embodiments, the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid tris(hydroxymethyl)aminomethane salt (1:1). In some other such embodiments, the GLP1R agonist is a combination of any of the above.

Administration may be carried out by any suitable delivery means, including, but not limited to, subcutaneous delivery, intravenous delivery, intramuscular delivery, intracisternal delivery, delivery by infusion techniques, transdermal delivery, oral delivery, nasal delivery, and rectal delivery. In some embodiments of any of the aforementioned embodiments, the administering comprises orally administering the GLP1R agonist. Suitable oral dosage forms are described in further detail below.

The disclosed methods may be carried out on any suitable subjects, including humans, horses, cows, sheep, pigs, mice, rats, dogs, cats, and primates such as chimpanzees, gorillas, and rhesus monkeys. In some embodiments of any of the aforementioned embodiments, the subject is a human. In the methods disclosed herein, the subject is a subject in need of the administration of the GLP1R agonist. The nature of the need depends on the therapeutic goals. In some embodiments of any of the foregoing embodiments, the subject exhibits elevated levels of glycated hemoglobin in its blood, for example, elevated levels of HbA1c in its blood. In some such embodiments, administering the GLP1R agonist is carried out to reduce the subject's HbA1c levels. In some other embodiments of any of the foregoing embodiments, the subject exhibits one or more symptoms consistent with type 2 diabetes. In some such embodiments, administering the GLP1R agonist is carried out to treat the type 2 diabetes or type 1 diabetes (including treating one or more of the symptoms associated therewith). In some other embodiments of any of the foregoing embodiments, the subject has elevated body mass, or in some cases, obesity. In some such embodiments, administering the GLP1R agonist is carried out to reduce body mass, treat obesity (including treating one or more of the symptoms associated therewith), or delay gastric emptying. In some other embodiments of any of the foregoing embodiments, the subject exhibits one or more symptoms consistent with poor glycemic control. In some such embodiments, administering the GLP1R agonist is carried out to improving glycemic control (including treating one or more of the symptoms associated therewith).

As noted above, the methods include administering from 0.1 to 5.0 mg/kg daily of the GLP1R agonist. These quantities may be administered in any suitable regimen throughout the day. In some embodiments, the administering comprises administering the GLP1R agonist one or more times a day, such as one time a day, two times a day, three times a day, and the like. In some further such embodiments, the administering comprises administering the GLP1R agonist two times a day. The administering may occur with or without food. In some embodiments wherein the administering comprises administering the GLP1R agonist one or more times a day, at least one of the one or more times is with food. In some such embodiments, the administering comprises administering the GLP1R agonist two times a day with food. In some embodiments, the two or more daily doses contain equal amounts of the GLP1R agonist. In other embodiments, the methods include administering from 0.1 to 5.0 mg/kg every other day of the GLP1R agonist, or every third day, or every fourth day, or every fifth day, or every sixth day.

The duration of the methods disclosed herein may be carried out over any suitable period of time, depending on treatment goals. Because type 2 diabetes or type 1 diabetes and its related disorders are chronic conditions, the administering may, in some embodiments, be carried out indefinitely, such as for several years or more. In some embodiments of any of the foregoing embodiments, the administering comprises administering the GLP1R agonist for a period of time no less than one week, or no less than two weeks, or no less than three weeks, or no less than six weeks, or no less than nine weeks, or no less than twelve weeks.

In some embodiments of any of the foregoing aspects and embodiments, the GLP1R agonist can be co-administered with one or more other antidiabetic agents in combination with the GLP1R agonist. In this context, the terms “coadministering” and “in combination with” do not necessarily imply that the antidiabetic agents are administered on the same schedule as the GLP1R agonist. After all, in some instances, these medications may be once-daily or once-weekly medications. Thus, in this context, the terms “coadministering” and “in combination with” refer to administering the drugs in such a way that the one or more antidiabetic agents have a non-zero concentration in the blood of the subject at the time of administering the GLP1R agonist. In some embodiments, the GLP1R agonist and one or more antidiabetic agents are formulated into the same dosage form, such as a tablet or capsule for oral administration.

Any suitable antidiabetic agents can be used. For example, in some embodiments, the one or more antidiabetic agents are selected from the group consisting of: insulin, insulin analogs (including insulin lispro, insulin aspart, insulin glulisine, isophane insulin, insulin zinc, insulin glargine, and insulin detemir), biguanides (including metformin, phenformin, and buformin), thiazolidinediones (including rosiglitazone, pioglitazone, and troglitazone), sulfonylureas (including tolbutamide, acetohexamide, tolazamide, chlorpropamide, glipizide, glibenclamide, glimepiride, gliclazide, glyclopyramide, and gliquidone), meglitinides (including repaglinide and nateglinide), alpha-glucosidase inhibitors (including miglitol, acarbose, and voglibose), glucagon-like peptide analogs and agonists (including exenatide, liraglutide, semaglutide, taspoglutide, lixisenatide, albuglutide, and dulaglutide), gastric inhibitory peptide analogs, dipeptidyl peptidase-4 (DPP-4) inhibitors (including vildagliptin, sitagliptin, saxagliptin, linagliptin, alogliptin, septagliptin, teneligliptin, and gemigliptin), amylin agonist analogs, sodium/glucose cotransporter 2 (SGLT2) inhibitors, and glucokinase activators. In some such embodiments, the one or more antidiabetic agents is metformin.

In embodiments where metformin is coadministered in combination with the GLP1R agonist, the coadministering comprises orally coadministering from 1 to 30 mg/kg daily of metformin to the subject or coadministering between 1 mg to 2,500 mg daily of metformin to the subject. This coadministering can occur in any suitable dosages. In some embodiments, the coadministering comprises coadministering metformin one or more times a day, such as one time a day, two times a day, three times a day, four times a day, and the like. In some such embodiments, the coadministering comprises coadministering metformin two times a day. In some further such embodiments, the coadministering comprises coadministering metformin two times a day with food. In some embodiments, the two or more daily doses contain equal amounts of metformin.

As noted above, in certain aspects and embodiments, administering the GLP1R agonist comprises administering to a subject in need thereof from 0.1 to 5.0 mg/kg daily.

In some further embodiments of any of the foregoing aspects and embodiments, administering the GLP1R agonist comprises administering to a subject in need thereof the GLP1R agonist (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing, in an amount from 0.1 to 2.5 mg/kg daily, or from 0.1 to 2.0 mg/kg daily, or from 0.1 to 1.7 mg/kg daily, or from 0.1 to 1.5 mg/kg daily, or from 0.3 to 3.0 mg/kg daily, or from 0.3 to 2.5 mg/kg daily, or from 0.3 to 2.0 mg/kg daily, or from 0.3 to 1.7 mg/kg daily, or from 0.3 to 1.5 mg/kg daily, or from 0.5 to 3.0 mg/kg daily, or from 0.5 to 2.5 mg/kg daily, or from 0.5 to 2.0 mg/kg daily, or from 0.5 to 1.7 mg/kg daily, or from 0.5 to 1.5 mg/kg daily, of the GLP1R agonist. In other embodiments, these ranges of GLP1R agonist are administered every other day, or every third day, or every fourth day, or every fifth day, or every sixth day.

In some other such embodiments, where the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing, administering the GLP1R agonist comprises administering to a human subject in need thereof from 10 to 200 mg daily, or from 10 to 175 mg daily, or from 10 to 150 mg daily, or from 10 to 125 mg daily, or from 10 to 100 mg daily, or from 20 to 200 mg daily, or from 20 to 175 mg daily, or from 20 to 150 mg daily, or from 20 to 125 mg daily, or from 20 to 100 mg daily, or from 30 to 200 mg daily, or from 30 to 175 mg daily, or from 30 to 150 mg daily, or from 30 to 125 mg daily, or from 30 to 100 mg daily, of the GLP1R agonist. In other embodiments, these amounts of GLP1R agonist are administered every other day, or every third day, or every fourth day, or every fifth day, or every sixth day.

In some further embodiments of any of the foregoing aspects and embodiments, administering the GLP1R agonist comprises administering to a subject in need thereof the GLP1R agonist (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing, in an amount from 1.0 to 5.0 mg/kg daily, or from 1.1 to 4.9 mg/kg daily, or from 1.2 to 4.8 mg/kg daily, or from 1.3 to 4.7 mg/kg daily, or from 1.4 to 4.6 mg/kg daily, or from 1.5 to 4.5 mg/kg daily, or from 2.0 to 4.5 mg/kg daily, or from 2.1 to 4.5 mg/kg daily, or from 2.2 to 4.5 mg/kg daily, or from 2.3 to 4.5 mg/kg daily, or from 2.8 to 4.5 mg/kg daily, or from 2.8 to 4.1 mg/kg daily, or from 2.8 to 4.0 mg/kg daily of the GLP1R agonist. In other embodiments, these ranges of GLP1R agonist are administered every other day, or every third day, or every fourth day, or every fifth day, or every sixth day.

In some other such embodiments, where the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing, administering the GLP1R agonist comprises administering to a human subject in need thereof from 25 to 450 mg daily, or from 50 to 425 mg daily, or from 50 to 400 mg daily, or from 200 to 400 mg daily, or from 250 to 450 mg daily, or from 250 to 350 mg daily, or from 50 to 350 mg daily, or from 50 to 300 mg daily, or from 100 to 300 mg daily, of the GLP1R agonist. In other embodiments, these amounts of GLP1R agonist are administered every other day, or every third day, or every fourth day, or every fifth day, or every sixth day.

As noted above, in certain aspects and embodiments, administering metformin comprises coadministering to a subject in need thereof from 1 to 30 mg/kg daily. In some further embodiments of any of the foregoing aspects and embodiments, coadministering metformin comprises administering to a subject in need thereof from 1 to 25 mg/kg daily, or from 1 to 20 mg/kg daily, or from 1 to 18 mg/kg daily, or from 1 to 16 mg/kg daily, or from 3 to 25 mg/kg daily, or from 3 to 20 mg/kg daily, or from 3 to 18 mg/kg daily, or from 3 to 16 mg/kg daily, or from 5 to 25 mg/kg daily, or from 5 to 20 mg/kg daily, or from 5 to 18 mg/kg daily, or from 5 to 16 mg/kg daily, of metformin. In some other such embodiments, coadministering metformin comprises coadministering to a human subject in need thereof from 100 to 1500 mg daily, or from 100 to 1400 mg daily, or from 100 to 1300 mg daily, or from 100 to 1200 mg daily, or from 200 to 1500 mg daily, or from 200 to 1400 mg daily, or from 200 to 1300 mg daily, or from 200 to 1200 mg daily, or from 300 to 1500 mg daily, or from 300 to 1400 mg daily, or from 300 to 1300 mg daily, or from 300 to 1200 mg daily, of metformin.

### Uses for Lowering Glycated Hemoglobin

The foregoing methods are set forth as general methods. In some embodiments of any of the foregoing aspects and embodiments, the methods are methods of lowering glycated hemoglobin levels in a subject. In some further such embodiments, lowering glycated hemoglobin levels comprises lowering HbA1c levels in a subject. For example, in some embodiments, lowering glycated hemoglobin levels comprises lowering HbA1c levels in a subject by an absolute amount of at least 0.3%, or an absolute amount of at least 0.5%, or an absolute amount of at least 0.7%, or an absolute amount of at least 0.9%, or an absolute amount of at least 1.0%, where HbA1c levels are measured as a percentage according to the National Glycohemoglobin Standardization Program (NGSP) protocol.

In some other embodiments of any of the foregoing aspects and embodiments, the disclosure provides GLP1R agonists for use in lowering elevated glycated hemoglobin levels in a subject according to any of the embodiments set forth above. In some other embodiments of any of the foregoing aspects and embodiments, the disclosure provides uses of GLP1R agonists in the manufacture of a medicament for lowering elevated levels of glycated hemoglobin in a subject, wherein the medicament is prepared to be administered to a subject according to any of the methods set forth above.

### Uses for Treating Type 2 Diabetes

The foregoing methods are set forth as general methods. In some embodiments of any of the foregoing aspects and embodiments, the methods are methods of treating type 2 diabetes.

In some other embodiments of any of the foregoing aspects and embodiments, the disclosure provides GLP1R agonists for use in treating type 2 diabetes according to any of the embodiments set forth above. In some other embodiments of any of the foregoing aspects and embodiments, the disclosure provides uses of GLP1R agonists in the manufacture of a medicament for treating type 2 diabetes, wherein the medicament is prepared to be administered to a subject according to any of the methods set forth above.

### Uses for Treating Obesity, Reducing Body Mass, or Delaying Gastric Emptying

The foregoing methods are set forth as general methods. In some embodiments of any of the foregoing aspects and embodiments, the methods are methods of treating obesity or methods of reducing body weight or mass or methods of delaying gastric emptying.

In some other embodiments of any of the foregoing aspects and embodiments, the disclosure provides GLP1R agonists for use in treating obesity or reducing body weight or mass or delaying gastric emptying according to any of the embodiments set forth above. In some other embodiments of any of the foregoing aspects and embodiments, the disclosure provides uses of GLP1R agonists in the manufacture of a medicament for treating obesity or reducing body weight or mass or delaying gastric emptying, wherein the medicament is prepared to be administered to a subject according to any of the methods set forth above.

### Uses for Improving Glycemic Control

The foregoing methods are set forth as general methods. In some embodiments of any of the foregoing aspects and embodiments, the methods are methods of improving glycemic control.

In some other embodiments of any of the foregoing aspects and embodiments, the disclosure provides GLP1R agonists for use in improving glycemic control according to any of the embodiments set forth above. In some other embodiments of any of the foregoing aspects and embodiments, the disclosure provides uses of GLP1R agonists in the manufacture of a medicament for improving glycemic control, wherein the medicament is prepared to be administered to a subject according to any of the methods set forth above.

### Uses for Lowering Fasting Glucose

The foregoing methods are set forth as general methods. In some embodiments of any of the foregoing aspects and embodiments, the methods are methods of lowering fasting plasma glucose (FPG), for example, to a subject in need thereof, such as a subject with elevated FPG.

In some other embodiments of any of the foregoing aspects and embodiments, the disclosure provides GLP1R agonists for use in lowering FPG according to any of the embodiments set forth above. In some other embodiments of any of the foregoing aspects and embodiments, the disclosure provides uses of GLP1R agonists in the manufacture of a medicament for lowering FPG, wherein the medicament is prepared to be administered to a subject according to any of the methods set forth above.

### Uses for Lowering Systolic Blood Pressure

The foregoing methods are set forth as general methods. In some embodiments of any of the foregoing aspects and embodiments, the methods are methods of lowering systolic blood pressure, for example, to a subject in need thereof, such as a subject with elevated systolic blood pressure.

In some other embodiments of any of the foregoing aspects and embodiments, the disclosure provides GLP1R agonists for use in lowering systolic blood pressure according to any of the embodiments set forth above. In some other embodiments of any of the foregoing aspects and embodiments, the disclosure provides uses of GLP1R agonists in the manufacture of a medicament for lowering systolic blood pressure, wherein the medicament is prepared to be administered to a subject according to any of the methods set forth above.

### Pharmaceutical Compositions Dosage Forms

The GLP1R agonists can be formulated into any suitable pharmaceutical composition. As used herein, the term “pharmaceutical composition” refers to a composition (e.g., a granulated powder or a liquid) that contains a pharmaceutically active ingredient (e.g., a GLP1R agonist) and a pharmaceutically acceptable carrier. As used herein, the term “pharmaceutically acceptable” refers to a substance that is not generally biologically undesirable at the administered quantities. In some embodiments, the GLP1R agonist is included in separate pharmaceutical composition from any coadministered antidiabetic agents (such as metformin), each of which also includes a pharmaceutically acceptable carrier. In other embodiments, the GLP1R agonist is included in the same pharmaceutical composition with one or more coadministered antidiabetic agents (such as metformin), which also includes a pharmaceutically acceptable carrier.

The pharmaceutical compositions, described herein, can be packaged in a form for oral administration as discrete units (i.e., dosage forms), such as capsules, tablets, sachets, or the like. Preparation of the solid compositions in forms intended for oral administration is within the ability of one skilled in the art, including the selection of pharmaceutically acceptable additional ingredients from the groups listed above in order to provide pharmaceutically elegant and palatable preparations. Such pharmaceutical compositions may be prepared by methods known in the pharmaceutical formulation art, for example, see Remington's Pharmaceutical Sciences, 18th Ed., (Mack Publishing Company, Easton, Pa., 1990).

### Embodiments

In addition to, or in further exemplification of, the various aspects and embodiments set forth above, the disclosure provides methods, uses, and the like as set forth in the embodiments below.

Embodiment 1. A method of lowering glycated hemoglobin levels in a subject, the method comprising administering to a subject in need thereof from 0.1 to 5.0 mg/kg or between 10 mg and 500 mg daily of a glucagon-like peptide 1 receptor (GLP1R) agonist.

Embodiment 2. The method of embodiment 1, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing.

Embodiment 3. The method of embodiment 2, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid.

Embodiment 4. The method of embodiment 2, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

Embodiment 5. The method of embodiment 2, wherein the GLP1R agonist is a combination of (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, and (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

Embodiment 6. The method of embodiment 1, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing.

Embodiment 7. The method of embodiment 6, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-proionic acid.

Embodiment 8. The method of embodiment 6, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 9. The method of embodiment 6, wherein the GLP1R agonist is a combination of (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, and (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 10. The method of any one of embodiments 1 to 9, wherein the administering comprises orally administering the GLP1R agonist.

Embodiment 11. The method of any one of embodiments 1 to 10, wherein the GLP1R agonist functions primarily as a GLP1R agonist at the administered dose.

Embodiment 12. The method of any one of embodiments 1 to 11, wherein the subject is a human.

Embodiment 13. The method of any one of embodiments 1 to 12, wherein the administering comprises administering the GLP1R agonist one or more times a day, such as one time a day, two times a day, three times a day, and the like.

Embodiment 14. The method of embodiment 13, wherein at least one of the one or more times is with food.

Embodiment 15. The method of embodiment 13, wherein the administering comprises administering the GLP1R agonist two times a day.

Embodiment 16. The method of embodiment 15, wherein the administering comprises administering the GLP1R agonist two times a day with food.

Embodiment 17. The method of any one of embodiments 1 to 16, wherein the administering comprises administering the GLP1R agonist for a period of time no less than one week, or no less than two weeks, or no less than three weeks, or no less than six weeks, or no less than nine weeks, or no less than twelve weeks.

Embodiment 18. The method of any one of embodiments 1 to 17, further comprising coadministering to the subject one or more antidiabetic agents in combination with the GLP1R agonist.

Embodiment 19. The method of embodiment 18, wherein the one or more antidiabetic agents are selected from the group consisting of: insulin, insulin analogs (including insulin lispro, insulin aspart, insulin glulisine, isophane insulin, insulin zinc, insulin glargine, and insulin detemir), biguanides (including metformin, phenformin, and buformin), thiazolidinediones (including rosiglitazone, pioglitazone, and troglitazone), sulfonylureas (including tolbutamide, acetohexamide, tolazamide, chlorpropamide, glipizide, glibenclamide, glimepiride, gliclazide, glyclopyramide, and gliquidone), meglitinides (including repaglinide and nateglinide), alpha-glucosidase inhibitors (including miglitol, acarbose, and voglibose), glucagon-like peptide analogs and agonists (including exenatide, liraglutide, semaglutide, taspoglutide, lixisenatide, albuglutide, and dulaglutide), gastric inhibitory peptide analogs, dipeptidyl peptidase-4 (DPP-4) inhibitors (including vildagliptin, sitagliptin, saxagliptin, linagliptin, alogliptin, septagliptin, teneligliptin, and gemigliptin), amylin agonist analogs, sodium/glucose cotransporter 2 (SGLT2) inhibitors, and glucokinase activators.

Embodiment 20. The method of embodiment 19, wherein the one or more antidiabetic agents is metformin.

Embodiment 21. The method of embodiment 20, wherein the coadministering comprises orally coadministering from 1 to 30 mg/kg daily of metformin to the subject.

Embodiment 22. The method of embodiment 21, wherein the coadministering comprises coadministering metformin one or more times a day, such as one time a day, two times a day, three times a day, four times a day, and the like.

Embodiment 23. The method of embodiment 22, wherein the coadministering comprises coadministering metformin two times a day.

Embodiment 24. The method of embodiment 23, wherein the coadministering comprises coadministering metformin two times a day with food.

Embodiment 25. The method of any one of embodiments 1 to 24, wherein administering the GLP1R agonist comprises administering to a subject in need thereof from 0.1 to 2.5 mg/kg daily, or from 0.1 to 2.0 mg/kg daily, or from 0.1 to 1.7 mg/kg daily, or from 0.1 to 1.5 mg/kg daily, or from 0.3 to 3.0 mg/kg daily, or from 0.3 to 2.5 mg/kg daily, or from 0.3 to 2.0 mg/kg daily, or from 0.3 to 1.7 mg/kg daily, or from 0.3 to 1.5 mg/kg daily, or from 0.5 to 3.0 mg/kg daily, or from 0.5 to 2.5 mg/kg daily, or from 0.5 to 2.0 mg/kg daily, or from 0.5 to 1.7 mg/kg daily, or from 0.5 to 1.5 mg/kg daily, of the GLP1R agonist

Embodiment 26. The method of any one of embodiments 1 to 25, wherein administering the GLP1R agonist comprises administering to a human subject in need thereof from 10 to 200 mg daily, or from 10 to 175 mg daily, or from 10 to 150 mg daily, or from 10 to 125 mg daily, or from 10 to 100 mg daily, or from 20 to 200 mg daily, or from 20 to 175 mg daily, or from 20 to 150 mg daily, or from 20 to 125 mg daily, or from 20 to 100 mg daily, or from 30 to 200 mg daily, or from 30 to 175 mg daily, or from 30 to 150 mg daily, or from 30 to 125 mg daily, or from 30 to 100 mg daily, of the GLP1R agonist.

Embodiment 27. The method of any one of embodiments 1 to 26, wherein coadministering metformin comprises administering to a subject in need thereof from 1 to 25 mg/kg daily, or from 1 to 20 mg/kg daily, or from 1 to 18 mg/kg daily, or from 1 to 16 mg/kg daily, or from 3 to 25 mg/kg daily, or from 3 to 20 mg/kg daily, or from 3 to 18 mg/kg daily, or from 3 to 16 mg/kg daily, or from 5 to 25 mg/kg daily, or from 5 to 20 mg/kg daily, or from 5 to 18 mg/kg daily, or from 5 to 16 mg/kg daily, of metformin.

Embodiment 28. The method of any one of embodiments 1 to 27, wherein coadministering metformin comprises coadministering to a human subject in need thereof from 100 to 1500 mg daily, or from 100 to 1400 mg daily, or from 100 to 1300 mg daily, or from 100 to 1200 mg daily, or from 200 to 1500 mg daily, or from 200 to 1400 mg daily, or from 200 to 1300 mg daily, or from 200 to 1200 mg daily, or from 300 to 1500 mg daily, or from 300 to 1400 mg daily, or from 300 to 1300 mg daily, or from 300 to 1200 mg daily, of metformin.

Embodiment 29. The method of any one of embodiments 1 to 28, wherein lowering glycated hemoglobin levels comprises lowering HbA1c levels in a subject.

Embodiment 30. The method of embodiment 29, wherein lowering glycated hemoglobin levels comprises lowering HbA1c levels in a subject by an absolute amount of at least 0.3%, or an absolute amount of at least 0.5%, or an absolute amount of at least 0.7%, or an absolute amount of at least 0.9%, or an absolute amount of at least 1.0%, where HbA1c levels are measured as a percentage according to the National Glycohemoglobin Standardization Program (NGSP) protocol.

Embodiment 31. A method of treating type 2 diabetes, the method comprising administering to a subject in need thereof from 0.1 to 5.0 mg/kg or between 10 mg and 500 mg daily of a glucagon-like peptide 1 receptor (GLP1R) agonist.

Embodiment 32. The method of embodiment 31, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing.

Embodiment 33. The method of embodiment 32, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid.

Embodiment 34. The method of embodiment 32, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

Embodiment 35. The method of embodiment 32, wherein the GLP1R agonist is a combination of (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, and (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

Embodiment 36. The method of embodiment 31, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing.

Embodiment 37. The method of embodiment 36, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-proionic acid.

Embodiment 38. The method of embodiment 36, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 39. The method of embodiment 36, wherein the GLP1R agonist is a combination of (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, and (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 40. The method of any one of embodiments 31 to 39, wherein the administering comprises orally administering the GLP1R agonist.

Embodiment 41. The method of any one of embodiments 31 to 40, wherein the GLP1R agonist functions primarily as a GLP1R agonist at the administered dose.

Embodiment 42. The method of any one of embodiments 31 to 41, wherein the subject is a human.

Embodiment 43. The method of any one of embodiments 31 to 42, wherein the administering comprises administering the GLP1R agonist one or more times a day, such as one time a day, two times a day, three times a day, and the like.

Embodiment 44. The method of embodiment 43, wherein at least one of the one or more times is with food.

Embodiment 45. The method of embodiment 43, wherein the administering comprises administering the GLP1R agonist two times a day.

Embodiment 46. The method of embodiment 45, wherein the administering comprises administering the GLP1R agonist two times a day with food.

Embodiment 47. The method of any one of embodiments 31 to 46, wherein the administering comprises administering the GLP1R agonist for a period of time no less than one week, or no less than two weeks, or no less than three weeks, or no less than six weeks, or no less than nine weeks, or no less than twelve weeks.

Embodiment 48. The method of any one of embodiments 31 to 47, further comprising coadministering to the subject one or more antidiabetic agents in combination with the GLP1R agonist.

Embodiment 49. The method of embodiment 48, wherein the one or more antidiabetic agents are selected from the group consisting of: insulin, insulin analogs (including insulin lispro, insulin aspart, insulin glulisine, isophane insulin, insulin zinc, insulin glargine, and insulin detemir), biguanides (including metformin, phenformin, and buformin), thiazolidinediones (including rosiglitazone, pioglitazone, and troglitazone), sulfonylureas (including tolbutamide, acetohexamide, tolazamide, chlorpropamide, glipizide, glibenclamide, glimepiride, gliclazide, glyclopyramide, and gliquidone), meglitinides (including repaglinide and nateglinide), alpha-glucosidase inhibitors (including miglitol, acarbose, and voglibose), glucagon-like peptide analogs and agonists (including exenatide, liraglutide, semaglutide, taspoglutide, lixisenatide, albuglutide, and dulaglutide), gastric inhibitory peptide analogs, dipeptidyl peptidase-4 (DPP-4) inhibitors (including vildagliptin, sitagliptin, saxagliptin, linagliptin, alogliptin, septagliptin, teneligliptin, and gemigliptin), amylin agonist analogs, sodium/glucose cotransporter 2 (SGLT2) inhibitors, and glucokinase activators.

Embodiment 50. The method of embodiment 49, wherein the one or more antidiabetic agents is metformin.

Embodiment 51. The method of embodiment 50, wherein the coadministering comprises orally coadministering from 1 to 30 mg/kg daily of metformin to the subject.

Embodiment 52. The method of embodiment 51, wherein the coadministering comprises coadministering metformin one or more times a day, such as one time a day, two times a day, three times a day, four times a day, and the like.

Embodiment 53. The method of embodiment 52, wherein the coadministering comprises coadministering metformin two times a day.

Embodiment 54. The method of embodiment 53, wherein the coadministering comprises coadministering metformin two times a day with food.

Embodiment 55. The method of any one of embodiments 31 to 54, wherein administering the GLP1R agonist comprises administering to a subject in need thereof from 0.1 to 2.5 mg/kg daily, or from 0.1 to 2.0 mg/kg daily, or from 0.1 to 1.7 mg/kg daily, or from 0.1 to 1.5 mg/kg daily, or from 0.3 to 3.0 mg/kg daily, or from 0.3 to 2.5 mg/kg daily, or from 0.3 to 2.0 mg/kg daily, or from 0.3 to 1.7 mg/kg daily, or from 0.3 to 1.5 mg/kg daily, or from 0.5 to 3.0 mg/kg daily, or from 0.5 to 2.5 mg/kg daily, or from 0.5 to 2.0 mg/kg daily, or from 0.5 to 1.7 mg/kg daily, or from 0.5 to 1.5 mg/kg daily, of the GLP1R agonist

Embodiment 56. The method of any one of embodiments 31 to 55, wherein administering the GLP1R agonist comprises administering to a human subject in need thereof from 10 to 200 mg daily, or from 10 to 175 mg daily, or from 10 to 150 mg daily, or from 10 to 125 mg daily, or from 10 to 100 mg daily, or from 20 to 200 mg daily, or from 20 to 175 mg daily, or from 20 to 150 mg daily, or from 20 to 125 mg daily, or from 20 to 100 mg daily, or from 30 to 200 mg daily, or from 30 to 175 mg daily, or from 30 to 150 mg daily, or from 30 to 125 mg daily, or from 30 to 100 mg daily, of the GLP1R agonist.

Embodiment 57. The method of any one of embodiments 31 to 56, wherein coadministering metformin comprises administering to a subject in need thereof from 1 to 25 mg/kg daily, or from 1 to 20 mg/kg daily, or from 1 to 18 mg/kg daily, or from 1 to 16 mg/kg daily, or from 3 to 25 mg/kg daily, or from 3 to 20 mg/kg daily, or from 3 to 18 mg/kg daily, or from 3 to 16 mg/kg daily, or from 5 to 25 mg/kg daily, or from 5 to 20 mg/kg daily, or from 5 to 18 mg/kg daily, or from 5 to 16 mg/kg daily, of metformin.

Embodiment 58. The method of any one of embodiments 31 to 57, wherein coadministering metformin comprises coadministering to a human subject in need thereof from 100 to 1500 mg daily, or from 100 to 1400 mg daily, or from 100 to 1300 mg daily, or from 100 to 1200 mg daily, or from 200 to 1500 mg daily, or from 200 to 1400 mg daily, or from 200 to 1300 mg daily, or from 200 to 1200 mg daily, or from 300 to 1500 mg daily, or from 300 to 1400 mg daily, or from 300 to 1300 mg daily, or from 300 to 1200 mg daily, of metformin.

Embodiment 59. A method of lowering body weight, the method comprising administering to a subject in need thereof from 0.1 to 3.0 mg/kg or between 10 mg and 500 mg daily of a glucagon-like peptide 1 receptor (GLP1R) agonist.

Embodiment 60. The method of embodiment 59, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing.

Embodiment 61. The method of embodiment 60, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid.

Embodiment 62. The method of embodiment 60, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

Embodiment 63. The method of embodiment 60, wherein the GLP1R agonist is a combination of (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, and (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2). Embodiment 64. The method of embodiment 59, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing.

Embodiment 65. The method of embodiment 64, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-proionic acid.

Embodiment 66. The method of embodiment 64, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 67. The method of embodiment 64, wherein the GLP1R agonist is a combination of (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, and (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 68. The method of any one of embodiments 59 to 67, wherein the administering comprises orally administering the GLP1R agonist.

Embodiment 69. The method of any one of embodiments 59 to 68, wherein the GLP1R agonist functions primarily as a GLP1R agonist at the administered dose.

Embodiment 70. The method of any one of embodiments 59 to 69, wherein the subject is a human.

Embodiment 71. The method of any one of embodiments 59 to 70, wherein the administering comprises administering the GLP1R agonist one or more times a day, such as one time a day, two times a day, three times a day, and the like.

Embodiment 72. The method of embodiment 71, wherein at least one of the one or more times is with food.

Embodiment 73. The method of embodiment 71, wherein the administering comprises administering the GLP1R agonist two times a day.

Embodiment 74. The method of embodiment 73, wherein the administering comprises administering the GLP1R agonist two times a day with food.

Embodiment 75. The method of any one of embodiments 59 to 74, wherein the administering comprises administering the GLP1R agonist for a period of time no less than one week, or no less than two weeks, or no less than three weeks, or no less than six weeks, or no less than nine weeks, or no less than twelve weeks.

Embodiment 76. The method of any one of embodiments 59 to 75, further comprising coadministering to the subject one or more antidiabetic agents in combination with the GLP1R agonist.

Embodiment 77. The method of embodiment 76, wherein the one or more antidiabetic agents are selected from the group consisting of: insulin, insulin analogs (including insulin lispro, insulin aspart, insulin glulisine, isophane insulin, insulin zinc, insulin glargine, and insulin detemir), biguanides (including metformin, phenformin, and buformin), thiazolidinediones (including rosiglitazone, pioglitazone, and troglitazone), sulfonylureas (including tolbutamide, acetohexamide, tolazamide, chlorpropamide, glipizide, glibenclamide, glimepiride, gliclazide, glyclopyramide, and gliquidone), meglitinides (including repaglinide and nateglinide), alpha-glucosidase inhibitors (including miglitol, acarbose, and voglibose), glucagon-like peptide analogs and agonists (including exenatide, liraglutide, semaglutide, taspoglutide, lixisenatide, albuglutide, and dulaglutide), gastric inhibitory peptide analogs, dipeptidyl peptidase-4 (DPP-4) inhibitors (including vildagliptin, sitagliptin, saxagliptin, linagliptin, alogliptin, septagliptin, teneligliptin, and gemigliptin), amylin agonist analogs, sodium/glucose cotransporter 2 (SGLT2) inhibitors, and glucokinase activators.

Embodiment 78. The method of embodiment 77, wherein the one or more antidiabetic agents is metformin.

Embodiment 79. The method of embodiment 78, wherein the coadministering comprises orally coadministering from 1 to 30 mg/kg daily of metformin to the subject.

Embodiment 80. The method of embodiment 79, wherein the coadministering comprises coadministering metformin one or more times a day, such as one time a day, two times a day, three times a day, four times a day, and the like.

Embodiment 81. The method of embodiment 80, wherein the coadministering comprises coadministering metformin two times a day.

Embodiment 82. The method of embodiment 81, wherein the coadministering comprises coadministering metformin two times a day with food.

Embodiment 83. The method of any one of embodiments 59 to 82, wherein administering the GLP1R agonist comprises administering to a subject in need thereof from 0.1 to 2.5 mg/kg daily, or from 0.1 to 2.0 mg/kg daily, or from 0.1 to 1.7 mg/kg daily, or from 0.1 to 1.5 mg/kg daily, or from 0.3 to 3.0 mg/kg daily, or from 0.3 to 2.5 mg/kg daily, or from 0.3 to 2.0 mg/kg daily, or from 0.3 to 1.7 mg/kg daily, or from 0.3 to 1.5 mg/kg daily, or from 0.5 to 3.0 mg/kg daily, or from 0.5 to 2.5 mg/kg daily, or from 0.5 to 2.0 mg/kg daily, or from 0.5 to 1.7 mg/kg daily, or from 0.5 to 1.5 mg/kg daily, of the GLP1R agonist

Embodiment 84. The method of any one of embodiments 59 to 83, wherein administering the GLP1R agonist comprises administering to a human subject in need thereof from 10 to 200 mg daily, or from 10 to 175 mg daily, or from 10 to 150 mg daily, or from 10 to 125 mg daily, or from 10 to 100 mg daily, or from 20 to 200 mg daily, or from 20 to 175 mg daily, or from 20 to 150 mg daily, or from 20 to 125 mg daily, or from 20 to 100 mg daily, or from 30 to 200 mg daily, or from 30 to 175 mg daily, or from 30 to 150 mg daily, or from 30 to 125 mg daily, or from 30 to 100 mg daily, of the GLP1R agonist.

Embodiment 85. The method of any one of embodiments 59 to 84, wherein coadministering metformin comprises administering to a subject in need thereof from 1 to 25 mg/kg daily, or from 1 to 20 mg/kg daily, or from 1 to 18 mg/kg daily, or from 1 to 16 mg/kg daily, or from 3 to 25 mg/kg daily, or from 3 to 20 mg/kg daily, or from 3 to 18 mg/kg daily, or from 3 to 16 mg/kg daily, or from 5 to 25 mg/kg daily, or from 5 to 20 mg/kg daily, or from 5 to 18 mg/kg daily, or from 5 to 16 mg/kg daily, of metformin.

Embodiment 86. The method of any one of embodiments 59 to 85, wherein coadministering metformin comprises coadministering to a human subject in need thereof from 100 to 1500 mg daily, or from 100 to 1400 mg daily, or from 100 to 1300 mg daily, or from 100 to 1200 mg daily, or from 200 to 1500 mg daily, or from 200 to 1400 mg daily, or from 200 to 1300 mg daily, or from 200 to 1200 mg daily, or from 300 to 1500 mg daily, or from 300 to 1400 mg daily, or from 300 to 1300 mg daily, or from 300 to 1200 mg daily, of metformin. Embodiment 87. A method of treating obesity, the method comprising administering to a subject in need thereof from 0.1 to 3.0 mg/kg or between 10 mg and 500 mg daily of a glucagon-like peptide 1 receptor (GLP1R) agonist.

Embodiment 88. The method of embodiment 87, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing.

Embodiment 89. The method of embodiment 88, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid.

Embodiment 90. The method of embodiment 88, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

Embodiment 91. The method of embodiment 88, wherein the GLP1R agonist is a combination of (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, and (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

Embodiment 92. The method of embodiment 87, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing.

Embodiment 93. The method of embodiment 92, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-proionic acid.

Embodiment 94. The method of embodiment 92, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 95. The method of embodiment 92, wherein the GLP1R agonist is a combination of (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, and (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 96. The method of any one of embodiments 87 to 95, wherein the administering comprises orally administering the GLP1R agonist.

Embodiment 97. The method of any one of embodiments 87 to 96, wherein the GLP1R agonist functions primarily as a GLP1R agonist at the administered dose.

Embodiment 98. The method of any one of embodiments 87 to 97, wherein the subject is a human.

Embodiment 99. The method of any one of embodiments 87 to 98, wherein the administering comprises administering the GLP1R agonist one or more times a day, such as one time a day, two times a day, three times a day, and the like.

Embodiment 100. The method of embodiment 99, wherein at least one of the one or more times is with food.

Embodiment 101. The method of embodiment 99, wherein the administering comprises administering the GLP1R agonist two times a day.

Embodiment 102. The method of embodiment 101, wherein the administering comprises administering the GLP1R agonist two times a day with food.

Embodiment 103. The method of any one of embodiments 87 to 102, wherein the administering comprises administering the GLP1R agonist for a period of time no less than one week, or no less than two weeks, or no less than three weeks, or no less than six weeks, or no less than nine weeks, or no less than twelve weeks.

Embodiment 104. The method of any one of embodiments 87 to 103, further comprising coadministering to the subject one or more antidiabetic agents in combination with the GLP1R agonist.

Embodiment 105. The method of embodiment 104, wherein the one or more antidiabetic agents are selected from the group consisting of: insulin, insulin analogs (including insulin lispro, insulin aspart, insulin glulisine, isophane insulin, insulin zinc, insulin glargine, and insulin detemir), biguanides (including metformin, phenformin, and buformin), thiazolidinediones (including rosiglitazone, pioglitazone, and troglitazone), sulfonylureas (including tolbutamide, acetohexamide, tolazamide, chlorpropamide, glipizide, glibenclamide, glimepiride, gliclazide, glyclopyramide, and gliquidone), meglitinides (including repaglinide and nateglinide), alpha-glucosidase inhibitors (including miglitol, acarbose, and voglibose), glucagon-like peptide analogs and agonists (including exenatide, liraglutide, semaglutide, taspoglutide, lixisenatide, albuglutide, and dulaglutide), gastric inhibitory peptide analogs, dipeptidyl peptidase-4 (DPP-4) inhibitors (including vildagliptin, sitagliptin, saxagliptin, linagliptin, alogliptin, septagliptin, teneligliptin, and gemigliptin), amylin agonist analogs, sodium/glucose cotransporter 2 (SGLT2) inhibitors, and glucokinase activators.

Embodiment 106. The method of embodiment 105, wherein the one or more antidiabetic agents is metformin.

Embodiment 107. The method of embodiment 106, wherein the coadministering comprises orally coadministering from 1 to 30 mg/kg daily of metformin to the subject.

Embodiment 108. The method of embodiment 107, wherein the coadministering comprises coadministering metformin one or more times a day, such as one time a day, two times a day, three times a day, four times a day, and the like.

Embodiment 109. The method of embodiment 108, wherein the coadministering comprises coadministering metformin two times a day.

Embodiment 110. The method of embodiment 109, wherein the coadministering comprises coadministering metformin two times a day with food.

Embodiment 111. The method of any one of embodiments 87 to 110, wherein administering the GLP1R agonist comprises administering to a subject in need thereof from 0.1 to 2.5 mg/kg daily, or from 0.1 to 2.0 mg/kg daily, or from 0.1 to 1.7 mg/kg daily, or from 0.1 to 1.5 mg/kg daily, or from 0.3 to 3.0 mg/kg daily, or from 0.3 to 2.5 mg/kg daily, or from 0.3 to 2.0 mg/kg daily, or from 0.3 to 1.7 mg/kg daily, or from 0.3 to 1.5 mg/kg daily, or from 0.5 to 3.0 mg/kg daily, or from 0.5 to 2.5 mg/kg daily, or from 0.5 to 2.0 mg/kg daily, or from 0.5 to 1.7 mg/kg daily, or from 0.5 to 1.5 mg/kg daily, of the GLP1R agonist

Embodiment 112. The method of any one of embodiments 87 to 111, wherein administering the GLP1R agonist comprises administering to a human subject in need thereof from 10 to 200 mg daily, or from 10 to 175 mg daily, or from 10 to 150 mg daily, or from 10 to 125 mg daily, or from 10 to 100 mg daily, or from 20 to 200 mg daily, or from 20 to 175 mg daily, or from 20 to 150 mg daily, or from 20 to 125 mg daily, or from 20 to 100 mg daily, or from 30 to 200 mg daily, or from 30 to 175 mg daily, or from 30 to 150 mg daily, or from 30 to 125 mg daily, or from 30 to 100 mg daily, of the GLP1R agonist.

Embodiment 113. The method of any one of embodiments 87 to 112, wherein coadministering metformin comprises administering to a subject in need thereof from 1 to 25 mg/kg daily, or from 1 to 20 mg/kg daily, or from 1 to 18 mg/kg daily, or from 1 to 16 mg/kg daily, or from 3 to 25 mg/kg daily, or from 3 to 20 mg/kg daily, or from 3 to 18 mg/kg daily, or from 3 to 16 mg/kg daily, or from 5 to 25 mg/kg daily, or from 5 to 20 mg/kg daily, or from 5 to 18 mg/kg daily, or from 5 to 16 mg/kg daily, of metformin.

Embodiment 114. The method of any one of embodiments 87 to 113, wherein coadministering metformin comprises coadministering to a human subject in need thereof from 100 to 1500 mg daily, or from 100 to 1400 mg daily, or from 100 to 1300 mg daily, or from 100 to 1200 mg daily, or from 200 to 1500 mg daily, or from 200 to 1400 mg daily, or from 200 to 1300 mg daily, or from 200 to 1200 mg daily, or from 300 to 1500 mg daily, or from 300 to 1400 mg daily, or from 300 to 1300 mg daily, or from 300 to 1200 mg daily, of metformin.

Embodiment 115. A method of improving glycemic control, the method comprising administering to a subject in need thereof from 0.1 to 3.0 mg/kg or between 10 mg and 500 mg daily of a glucagon-like peptide 1 receptor (GLP1R) agonist.

Embodiment 116. The method of embodiment 115, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing.

Embodiment 117. The method of embodiment 116, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid.

Embodiment 118. The method of embodiment 116, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

Embodiment 119. The method of embodiment 116, wherein the GLP1R agonist is a combination of (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, and (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

Embodiment 120. The method of embodiment 115, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing.

Embodiment 121. The method of embodiment 120, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-proionic acid.

Embodiment 122. The method of embodiment 120, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 123. The method of embodiment 120, wherein the GLP1R agonist is a combination of (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, and (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 124. The method of any one of embodiments 115 to 123, wherein the administering comprises orally administering the GLP1R agonist.

Embodiment 125. The method of any one of embodiments 115 to 124, wherein the GLP1R agonist functions primarily as a GLP1R agonist at the administered dose.

Embodiment 126. The method of any one of embodiments 115 to 125, wherein the subject is a human.

Embodiment 127. The method of any one of embodiments 115 to 126, wherein the administering comprises administering the GLP1R agonist one or more times a day, such as one time a day, two times a day, three times a day, and the like.

Embodiment 128. The method of embodiment 127, wherein at least one of the one or more times is with food.

Embodiment 129. The method of embodiment 127, wherein the administering comprises administering the GLP1R agonist two times a day.

Embodiment 130. The method of embodiment 129, wherein the administering comprises administering the GLP1R agonist two times a day with food.

Embodiment 131. The method of any one of embodiments 115 to 130, wherein the administering comprises administering the GLP1R agonist for a period of time no less than one week, or no less than two weeks, or no less than three weeks, or no less than six weeks, or no less than nine weeks, or no less than twelve weeks.

Embodiment 132. The method of any one of embodiments 115 to 131, further comprising coadministering to the subject one or more antidiabetic agents in combination with the GLP1R agonist.

Embodiment 133. The method of embodiment 132, wherein the one or more antidiabetic agents are selected from the group consisting of: insulin, insulin analogs (including insulin lispro, insulin aspart, insulin glulisine, isophane insulin, insulin zinc, insulin glargine, and insulin detemir), biguanides (including metformin, phenformin, and buformin), thiazolidinediones (including rosiglitazone, pioglitazone, and troglitazone), sulfonylureas (including tolbutamide, acetohexamide, tolazamide, chlorpropamide, glipizide, glibenclamide, glimepiride, gliclazide, glyclopyramide, and gliquidone), meglitinides (including repaglinide and nateglinide), alpha-glucosidase inhibitors (including miglitol, acarbose, and voglibose), glucagon-like peptide analogs and agonists (including exenatide, liraglutide, semaglutide, taspoglutide, lixisenatide, albuglutide, and dulaglutide), gastric inhibitory peptide analogs, dipeptidyl peptidase-4 (DPP-4) inhibitors (including vildagliptin, sitagliptin, saxagliptin, linagliptin, alogliptin, septagliptin, teneligliptin, and gemigliptin), amylin agonist analogs, sodium/glucose cotransporter 2 (SGLT2) inhibitors, and glucokinase activators.

Embodiment 134. The method of embodiment 133, wherein the one or more antidiabetic agents is metformin.

Embodiment 135. The method of embodiment 134, wherein the coadministering comprises orally coadministering from 1 to 30 mg/kg daily of metformin to the subject.

Embodiment 136. The method of embodiment 135, wherein the coadministering comprises coadministering metformin one or more times a day, such as one time a day, two times a day, three times a day, four times a day, and the like.

Embodiment 137. The method of embodiment 136, wherein the coadministering comprises coadministering metformin two times a day.

Embodiment 138. The method of embodiment 137, wherein the coadministering comprises coadministering metformin two times a day with food.

Embodiment 139. The method of any one of embodiments 115 to 138, wherein administering the GLP1R agonist comprises administering to a subject in need thereof from 0.1 to 2.5 mg/kg daily, or from 0.1 to 2.0 mg/kg daily, or from 0.1 to 1.7 mg/kg daily, or from 0.1 to 1.5 mg/kg daily, or from 0.3 to 3.0 mg/kg daily, or from 0.3 to 2.5 mg/kg daily, or from 0.3 to 2.0 mg/kg daily, or from 0.3 to 1.7 mg/kg daily, or from 0.3 to 1.5 mg/kg daily, or from 0.5 to 3.0 mg/kg daily, or from 0.5 to 2.5 mg/kg daily, or from 0.5 to 2.0 mg/kg daily, or from 0.5 to 1.7 mg/kg daily, or from 0.5 to 1.5 mg/kg daily, of the GLP1R agonist Embodiment 140. The method of any one of embodiments 115 to 139, wherein administering the GLP1R agonist comprises administering to a human subject in need thereof from 10 to 200 mg daily, or from 10 to 175 mg daily, or from 10 to 150 mg daily, or from 10 to 125 mg daily, or from 10 to 100 mg daily, or from 20 to 200 mg daily, or from 20 to 175 mg daily, or from 20 to 150 mg daily, or from 20 to 125 mg daily, or from 20 to 100 mg daily, or from 30 to 200 mg daily, or from 30 to 175 mg daily, or from 30 to 150 mg daily, or from 30 to 125 mg daily, or from 30 to 100 mg daily, of the GLP1R agonist.

Embodiment 141. The method of any one of embodiments 115 to 140, wherein coadministering metformin comprises administering to a subject in need thereof from 1 to 25 mg/kg daily, or from 1 to 20 mg/kg daily, or from 1 to 18 mg/kg daily, or from 1 to 16 mg/kg daily, or from 3 to 25 mg/kg daily, or from 3 to 20 mg/kg daily, or from 3 to 18 mg/kg daily, or from 3 to 16 mg/kg daily, or from 5 to 25 mg/kg daily, or from 5 to 20 mg/kg daily, or from 5 to 18 mg/kg daily, or from 5 to 16 mg/kg daily, of metformin.

Embodiment 142. The method of any one of embodiments 115 to 141, wherein coadministering metformin comprises coadministering to a human subject in need thereof from 100 to 1500 mg daily, or from 100 to 1400 mg daily, or from 100 to 1300 mg daily, or from 100 to 1200 mg daily, or from 200 to 1500 mg daily, or from 200 to 1400 mg daily, or from 200 to 1300 mg daily, or from 200 to 1200 mg daily, or from 300 to 1500 mg daily, or from 300 to 1400 mg daily, or from 300 to 1300 mg daily, or from 300 to 1200 mg daily, of metformin.

Embodiment 143. A method of lowering fasting plasma glucose (FGP), the method comprising administering to a subject in need thereof from 0.1 to 3.0 mg/kg or between 10 mg and 500 mg daily of a glucagon-like peptide 1 receptor (GLP1R) agonist.

Embodiment 144. The method of embodiment 143, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing.

Embodiment 145. The method of embodiment 144, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid.

Embodiment 146. The method of embodiment 144, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

Embodiment 147. The method of embodiment 144, wherein the GLP1R agonist is a combination of (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, and (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2). Embodiment 148. The method of embodiment 143, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing.

Embodiment 149. The method of embodiment 148, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-proionic acid.

Embodiment 150. The method of embodiment 148, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 151. The method of embodiment 148, wherein the GLP1R agonist is a combination of (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, and (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 152. The method of any one of embodiments 143 to 151, wherein the administering comprises orally administering the GLP1R agonist.

Embodiment 153. The method of any one of embodiments 143 to 152, wherein the GLP1R agonist functions primarily as a GLP1R agonist at the administered dose.

Embodiment 154. The method of any one of embodiments 143 to 153, wherein the subject is a human.

Embodiment 155. The method of any one of embodiments 143 to 154, wherein the administering comprises administering the GLP1R agonist one or more times a day, such as one time a day, two times a day, three times a day, and the like.

Embodiment 156. The method of embodiment 155, wherein at least one of the one or more times is with food.

Embodiment 157. The method of embodiment 155, wherein the administering comprises administering the GLP1R agonist two times a day.

Embodiment 158. The method of embodiment 157, wherein the administering comprises administering the GLP1R agonist two times a day with food.

Embodiment 159. The method of any one of embodiments 143 to 158, wherein the administering comprises administering the GLP1R agonist for a period of time no less than one week, or no less than two weeks, or no less than three weeks, or no less than six weeks, or no less than nine weeks, or no less than twelve weeks.

Embodiment 160. The method of any one of embodiments 143 to 159, further comprising coadministering to the subject one or more antidiabetic agents in combination with the GLP1R agonist.

Embodiment 161. The method of embodiment 160, wherein the one or more antidiabetic agents are selected from the group consisting of: insulin, insulin analogs (including insulin lispro, insulin aspart, insulin glulisine, isophane insulin, insulin zinc, insulin glargine, and insulin detemir), biguanides (including metformin, phenformin, and buformin), thiazolidinediones (including rosiglitazone, pioglitazone, and troglitazone), sulfonylureas (including tolbutamide, acetohexamide, tolazamide, chlorpropamide, glipizide, glibenclamide, glimepiride, gliclazide, glyclopyramide, and gliquidone), meglitinides (including repaglinide and nateglinide), alpha-glucosidase inhibitors (including miglitol, acarbose, and voglibose), glucagon-like peptide analogs and agonists (including exenatide, liraglutide, semaglutide, taspoglutide, lixisenatide, albuglutide, and dulaglutide), gastric inhibitory peptide analogs, dipeptidyl peptidase-4 (DPP-4) inhibitors (including vildagliptin, sitagliptin, saxagliptin, linagliptin, alogliptin, septagliptin, teneligliptin, and gemigliptin), amylin agonist analogs, sodium/glucose cotransporter 2 (SGLT2) inhibitors, and glucokinase activators.

Embodiment 162. The method of embodiment 161, wherein the one or more antidiabetic agents is metformin.

Embodiment 163. The method of embodiment 162, wherein the coadministering comprises orally coadministering from 1 to 30 mg/kg daily of metformin to the subject.

Embodiment 164. The method of embodiment 163, wherein the coadministering comprises coadministering metformin one or more times a day, such as one time a day, two times a day, three times a day, four times a day, and the like.

Embodiment 165. The method of embodiment 164, wherein the coadministering comprises coadministering metformin two times a day.

Embodiment 166. The method of embodiment 165, wherein the coadministering comprises coadministering metformin two times a day with food.

Embodiment 167. The method of any one of embodiments 143 to 166, wherein administering the GLP1R agonist comprises administering to a subject in need thereof from 0.1 to 2.5 mg/kg daily, or from 0.1 to 2.0 mg/kg daily, or from 0.1 to 1.7 mg/kg daily, or from 0.1 to 1.5 mg/kg daily, or from 0.3 to 3.0 mg/kg daily, or from 0.3 to 2.5 mg/kg daily, or from 0.3 to 2.0 mg/kg daily, or from 0.3 to 1.7 mg/kg daily, or from 0.3 to 1.5 mg/kg daily, or from 0.5 to 3.0 mg/kg daily, or from 0.5 to 2.5 mg/kg daily, or from 0.5 to 2.0 mg/kg daily, or from 0.5 to 1.7 mg/kg daily, or from 0.5 to 1.5 mg/kg daily, of the GLP1R agonist

Embodiment 168. The method of any one of embodiments 143 to 167, wherein administering the GLP1R agonist comprises administering to a human subject in need thereof from 10 to 200 mg daily, or from 10 to 175 mg daily, or from 10 to 150 mg daily, or from 10 to 125 mg daily, or from 10 to 100 mg daily, or from 20 to 200 mg daily, or from 20 to 175 mg daily, or from 20 to 150 mg daily, or from 20 to 125 mg daily, or from 20 to 100 mg daily, or from 30 to 200 mg daily, or from 30 to 175 mg daily, or from 30 to 150 mg daily, or from 30 to 125 mg daily, or from 30 to 100 mg daily, of the GLP1R agonist.

Embodiment 169. The method of any one of embodiments 143 to 168, wherein coadministering metformin comprises administering to a subject in need thereof from 1 to 25 mg/kg daily, or from 1 to 20 mg/kg daily, or from 1 to 18 mg/kg daily, or from 1 to 16 mg/kg daily, or from 3 to 25 mg/kg daily, or from 3 to 20 mg/kg daily, or from 3 to 18 mg/kg daily, or from 3 to 16 mg/kg daily, or from 5 to 25 mg/kg daily, or from 5 to 20 mg/kg daily, or from 5 to 18 mg/kg daily, or from 5 to 16 mg/kg daily, of metformin.

Embodiment 170. The method of any one of embodiments 143 to 169, wherein coadministering metformin comprises coadministering to a human subject in need thereof from 100 to 1500 mg daily, or from 100 to 1400 mg daily, or from 100 to 1300 mg daily, or from 100 to 1200 mg daily, or from 200 to 1500 mg daily, or from 200 to 1400 mg daily, or from 200 to 1300 mg daily, or from 200 to 1200 mg daily, or from 300 to 1500 mg daily, or from 300 to 1400 mg daily, or from 300 to 1300 mg daily, or from 300 to 1200 mg daily, of metformin. Embodiment 171. The method of any one of embodiments 143 to 2170, wherein lowering FPG comprises lowering FPG in a subject having FPG of at least 100 mg/dL, or at least 110 mg/dL, or at least 120 mg/dL, or at least 130 mg/dL, or at least 140 mg/dL.

Embodiment 172. The method of any one of embodiments 143 to 171, wherein lowering FPG comprises lowering FPG in a subject by at least 10 mg/dL, or at least 20 mg/dL, or at least 30 mg/dL, or at least 40 mg/dL, following daily administration for a period of 12 weeks.

Embodiment 173. A method of lowering systolic blood pressure, the method comprising administering to a subject in need thereof from 0.1 to 3.0 mg/kg or between 10 mg and 500 mg daily of a glucagon-like peptide 1 receptor (GLP1R) agonist.

Embodiment 174. The method of embodiment 173, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing.

Embodiment 175. The method of embodiment 174, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid.

Embodiment 176. The method of embodiment 174, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

Embodiment 177. The method of embodiment 174, wherein the GLP1R agonist is a combination of (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, and (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

Embodiment 178. The method of embodiment 173, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, a mass-equivalent of a pharmaceutically acceptable salt thereof, or any combination of the foregoing.

Embodiment 179. The method of embodiment 178, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-proionic acid.

Embodiment 180. The method of embodiment 178, wherein the GLP1R agonist is (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 181. The method of embodiment 178, wherein the GLP1R agonist is a combination of (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid, and (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid hydrochloride (1:1).

Embodiment 182. The method of any one of embodiments 173 to 181, wherein the administering comprises orally administering the GLP1R agonist.

Embodiment 183. The method of any one of embodiments 173 to 182, wherein the GLP1R agonist functions primarily as a GLP1R agonist at the administered dose.

Embodiment 184. The method of any one of embodiments 173 to 183, wherein the subject is a human.

Embodiment 185. The method of any one of embodiments 173 to 184, wherein the administering comprises administering the GLP1R agonist one or more times a day, such as one time a day, two times a day, three times a day, and the like.

Embodiment 186. The method of embodiment 185, wherein at least one of the one or more times is with food.

Embodiment 187. The method of embodiment 185, wherein the administering comprises administering the GLP1R agonist two times a day.

Embodiment 188. The method of embodiment 187, wherein the administering comprises administering the GLP1R agonist two times a day with food.

Embodiment 189. The method of any one of embodiments 173 to 188, wherein the administering comprises administering the GLP1R agonist for a period of time no less than one week, or no less than two weeks, or no less than three weeks, or no less than six weeks, or no less than nine weeks, or no less than twelve weeks.

Embodiment 190. The method of any one of embodiments 173 to 189, further comprising coadministering to the subject one or more antidiabetic agents in combination with the GLP1R agonist.

Embodiment 191. The method of embodiment 190, wherein the one or more antidiabetic agents are selected from the group consisting of: insulin, insulin analogs (including insulin lispro, insulin aspart, insulin glulisine, isophane insulin, insulin zinc, insulin glargine, and insulin detemir), biguanides (including metformin, phenformin, and buformin), thiazolidinediones (including rosiglitazone, pioglitazone, and troglitazone), sulfonylureas (including tolbutamide, acetohexamide, tolazamide, chlorpropamide, glipizide, glibenclamide, glimepiride, gliclazide, glyclopyramide, and gliquidone), meglitinides (including repaglinide and nateglinide), alpha-glucosidase inhibitors (including miglitol, acarbose, and voglibose), glucagon-like peptide analogs and agonists (including exenatide, liraglutide, semaglutide, taspoglutide, lixisenatide, albuglutide, and dulaglutide), gastric inhibitory peptide analogs, dipeptidyl peptidase-4 (DPP-4) inhibitors (including vildagliptin, sitagliptin, saxagliptin, linagliptin, alogliptin, septagliptin, teneligliptin, and gemigliptin), amylin agonist analogs, sodium/glucose cotransporter 2 (SGLT2) inhibitors, and glucokinase activators.

Embodiment 192. The method of embodiment 191, wherein the one or more antidiabetic agents is metformin.

Embodiment 193. The method of embodiment 192, wherein the coadministering comprises orally coadministering from 1 to 30 mg/kg daily of metformin to the subject.

Embodiment 194. The method of embodiment 193, wherein the coadministering comprises coadministering metformin one or more times a day, such as one time a day, two times a day, three times a day, four times a day, and the like.

Embodiment 195. The method of embodiment 194, wherein the coadministering comprises coadministering metformin two times a day.

Embodiment 196. The method of embodiment 195, wherein the coadministering comprises coadministering metformin two times a day with food.

Embodiment 197. The method of any one of embodiments 173 to 196, wherein administering the GLP1R agonist comprises administering to a subject in need thereof from 0.1 to 2.5 mg/kg daily, or from 0.1 to 2.0 mg/kg daily, or from 0.1 to 1.7 mg/kg daily, or from 0.1 to 1.5 mg/kg daily, or from 0.3 to 3.0 mg/kg daily, or from 0.3 to 2.5 mg/kg daily, or from 0.3 to 2.0 mg/kg daily, or from 0.3 to 1.7 mg/kg daily, or from 0.3 to 1.5 mg/kg daily, or from 0.5 to 3.0 mg/kg daily, or from 0.5 to 2.5 mg/kg daily, or from 0.5 to 2.0 mg/kg daily, or from 0.5 to 1.7 mg/kg daily, or from 0.5 to 1.5 mg/kg daily, of the GLP1R agonist

Embodiment 198. The method of any one of embodiments 173 to 197, wherein administering the GLP1R agonist comprises administering to a human subject in need thereof from 10 to 200 mg daily, or from 10 to 175 mg daily, or from 10 to 150 mg daily, or from 10 to 125 mg daily, or from 10 to 100 mg daily, or from 20 to 200 mg daily, or from 20 to 175 mg daily, or from 20 to 150 mg daily, or from 20 to 125 mg daily, or from 20 to 100 mg daily, or from 30 to 200 mg daily, or from 30 to 175 mg daily, or from 30 to 150 mg daily, or from 30 to 125 mg daily, or from 30 to 100 mg daily, of the GLP1R agonist.

Embodiment 199. The method of any one of embodiments 173 to 198, wherein coadministering metformin comprises administering to a subject in need thereof from 1 to 25 mg/kg daily, or from 1 to 20 mg/kg daily, or from 1 to 18 mg/kg daily, or from 1 to 16 mg/kg daily, or from 3 to 25 mg/kg daily, or from 3 to 20 mg/kg daily, or from 3 to 18 mg/kg daily, or from 3 to 16 mg/kg daily, or from 5 to 25 mg/kg daily, or from 5 to 20 mg/kg daily, or from 5 to 18 mg/kg daily, or from 5 to 16 mg/kg daily, of metformin.

Embodiment 200. The method of any one of embodiments 173 to 199, wherein coadministering metformin comprises coadministering to a human subject in need thereof from 100 to 1500 mg daily, or from 100 to 1400 mg daily, or from 100 to 1300 mg daily, or from 100 to 1200 mg daily, or from 200 to 1500 mg daily, or from 200 to 1400 mg daily, or from 200 to 1300 mg daily, or from 200 to 1200 mg daily, or from 300 to 1500 mg daily, or from 300 to 1400 mg daily, or from 300 to 1300 mg daily, or from 300 to 1200 mg daily, of metformin.

Embodiment 201. The method of any one of embodiments 173 to 200, wherein lowering systolic blood pressure comprises lowering systolic blood pressure in a subject having systolic blood pressure of at least 130 mm Hg, or at least 135 mm Hg, or at least 140 mm Hg, or at least 145 mm Hg, or at least 150 mm Hg.

Embodiment 202. The method of any one of embodiments 173 to 201, wherein lowering systolic blood pressure comprises lowering systolic blood pressure in a subject by at least 2 mm Hg, or at least 3 mm Hg, or at least 4 mm Hg, following daily administration for a period of 12 weeks.

### EXAMPLES

### Tablet Dosage for API-1

Tablets containing 75 mg of (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2) (“API-1”) were prepared.

### Tablet Dosage for API-2

Tablets containing 200 mg of (S)-3-(4′-cyano-biphenyl-4-yl)-2-{[(3R,7S)-3-[4-(3,4-dichlorobenzyloxy)-phenyl]-1-methyl-2-oxo-6-((S)-1-phenyl-propyl)-2,3,5,6,7,8-hexahydro-1H-4-oxa-1,6-diaza-anthracene-7-carbonyl]-amino}-propionic acid as a 1:1 tris(hydroxymethyl)aminomethane salt (“API-2”) were prepared.

### Example 1—Phase 2 Study of API-1

A Phase 2 clinical study was designed as a randomized, double-blind, placebo-controlled, parallel-group multi-center study evaluating the efficacy and safety following 12 weeks of treatment with API-1 in subjects (humans) with type 2 diabetes who were being treated with a stable dose of metformin. Subjects were randomly assigned to one of three treatment groups, API-1 150 mg BID, API-1 150 mg QPM, or placebo with approximately n=52/group.

A total of approximately 156 patients were to be randomized into this study and 174 were ultimately randomized. Subjects were enrolled and randomized according to a fixed randomization scheme blocked by study investigative site. Severity of diabetes was determined by HbA1c measures (7.5% to 10%) at screening, confirmed at Baseline, and HbA1c was used as a stratification variable in the randomization scheme: HbA1c less than 8% and 8% or more. Randomization was to be a balanced allocation (1:1:1) among the three treatment groups. Dropouts were not to be replaced.

Statistical analyses for topline results included placebo-subtracted change from baseline (LSM-CFB) for HbA1c (primary endpoint) and body weight (secondary endpoint) as well as a post hoc concentration/effect analysis to evaluate the effect of exposure on efficacy.

The once and twice daily treatment arms of API-1 demonstrated placebo-subtracted decreases from baseline in HbA1c (%) at 12 weeks of (i) 0.9±0.2% (p<0.001) and 0.7±0.2% (p<0.001), respectively. Although the study was not powered to demonstrate weight loss, trends were observed at 12 weeks with patients losing on average 0.9 kg±0.5 kg (p=0.08) in the 150 mg once daily arm, and 0.6 kg±0.5 kg in the 150 mg twice daily arm. Further, the once and twice daily treatment arms of API-1 demonstrated placebo-subtracted decreases from baseline in fasting plasma glucose (mg/dL) at 12 weeks of about 16 and 17, respectively.

Subsequent concentration/effect analysis of completers revealed that lower doses demonstrated more pronounced effects for key efficacy endpoints such as, but not limited to, HbA1c, fasting plasma glucose (FGP), and weight loss. This observation is confirmed when correlating plasma concentration with efficacy. Patients dosed with lower than 1.35 mg/kg of API-1 had greater efficacy with reductions from baseline in HbA_{1c} and body weight of 1.7% (p<0.01) and 3.7 kg (p<0.05), respectively, at 12 weeks (See Table 3 below).

TABLE 3
Mean Change from Baseline HbA1c (%) for API-1
analyzed by dose/weight (mg/kg) by Visit

| | Concentration Group | | | | |
|---|---|---|---|---|---|
| | | 1-1.35 | | | 1.76-2.2 |
| | | mg/kg | 1.35-1.64 | 1.65-1.75 | mg/kg |
| Visit | Placebo | (<1.35) | mg/kg | mg/kg | (>1.75) |
| Week 4 | 0.16 | −0.57 | −0.35 | −0.21 | −0.31 |
| Week 8 | 0.09 | −1.26 | −0.85 | −0.51 | −0.43 |
| Week 12 | −0.03 | −1.71 | −0.90 | −0.75 | −0.55 |

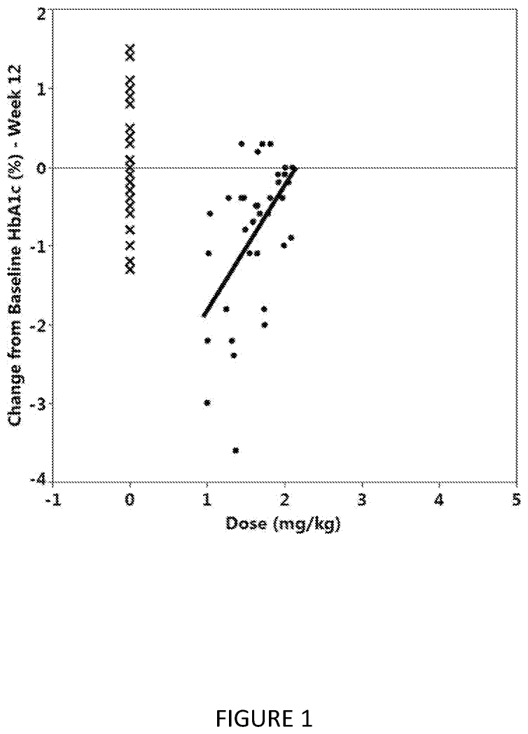

FIG. 1. Dose (mg/kg) response in HbA1c for 150 mg once daily (QPM) treatment arm: In FIG. 1, the data points represent the change from baseline at Week 12 by dose (mg/kg) for HbA1c (%) for completers by treatment arm (placebo=X; 150 mg once daily (QPM)=•). The line through the data points between 1 to 2.2 mg/kg represents the linear line of fit of these data points. The slope of the line through the data points for the 150 mg once daily (QPM) treatment arm shows that the change in HbA1c from baseline increased as the dose was reduced from 2.2 mg/kg to 1.0 mg/kg.

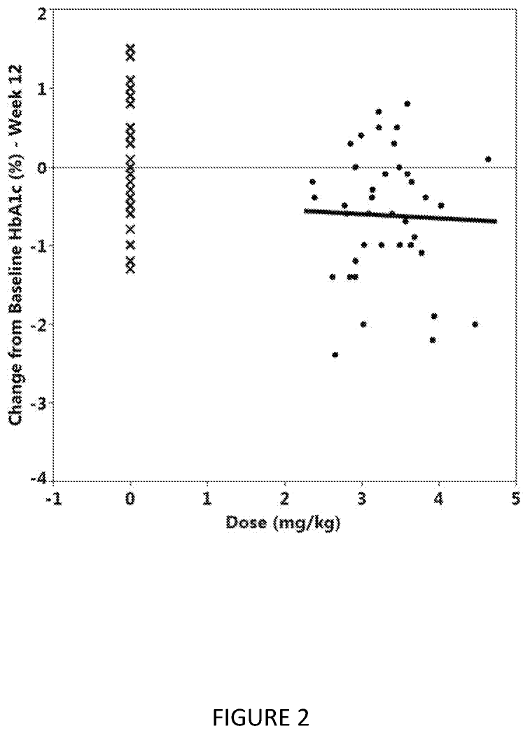

FIG. 2. Dose (mg/kg) response in HbA1c for 150 mg twice daily (BID) treatment arm: In FIG. 2, the data points represent the change from baseline at Week 12 by dose (mg/kg) for HbA1c (%) for completers by treatment arm (placebo=X; 150 mg twice daily (BID)=•). The line through the data points between 2.2 mg/kg and 4.8 mg/kg represents the linear line of fit of these data points. The slope of the line through the data points for the 150 mg twice daily (BID) treatment arm shows that the change in HbA1c from baseline was almost unchanged across the doses between 2.2 mg/kg and 4.8 mg/kg.

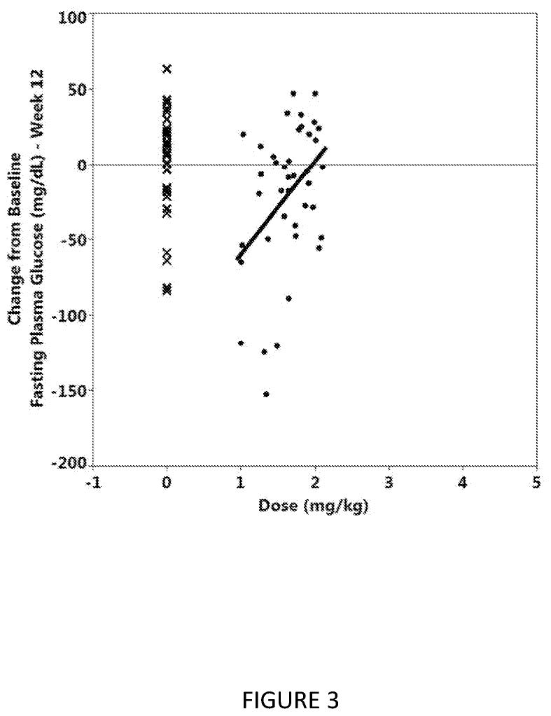

FIG. 3. Dose (mg/kg) response in FPG for 150 mg once daily (QPM) treatment arm: In FIG. 3, the data points represent the change from baseline at Week 12 by dose (mg/kg) for fasting plasma glucose (FPG) (mg/dL) for completers by treatment arm (placebo=X; 150 mg once daily (QPM)=•). The line through the data points between 1 to 2.2 mg/kg represents the linear line of fit of these data points. The slope of the line through the data points for the 150 mg once daily (QPM) treatment arm shows that the change in FPG from baseline increased as the dose was reduced from 2.2 mg/kg to 1.0 mg/kg.

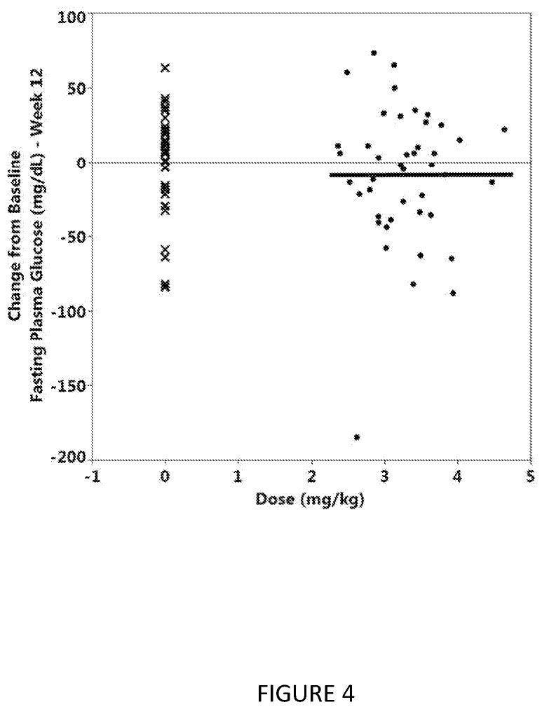

FIG. 4. Dose (mg/kg) response for in FPG 150 mg twice daily (BID) treatment arm: In FIG. 4, the data points represent the change from baseline at Week 12 by dose (mg/kg) for fasting plasma glucose (FPG) (mg/dL) for completers by treatment arm (placebo=X; 150 mg twice daily (BID)=•). The line through the data points between 2.2 mg/kg and 4.8 mg/kg represents the linear line of fit of these data points. The slope of the line through the data points for the 150 mg twice daily (BID) treatment arm shows that the change in FPG (mg/dL) from baseline was almost unchanged across the doses between 2.2 mg/kg and 4.8 mg/kg.

### Example 2—Phase 2 Study of API-2

A Phase 2 study was designed as a randomized, double-blind, placebo controlled, dose-ranging, parallel group study evaluating the efficacy and safety following 12 weeks of treatment with API-2 in subjects (humans) with T2DM. The study was conducted in 2 parts: Part A and Part B. Part A and Part B were identical except for the dose levels administered. In Part A, subjects received one of two treatments administered orally for 12 weeks: API-2 400 mg QD (n=51) or matching placebo (n=26). In Part B, subjects received one of three treatments administered orally for 12 weeks: API-2 200 mg QD (n=28), API-2 800 mg QD (n=56), or matching placebo (n=26).

TABLE 4
Mean Change from Baseline in HbA1c (%) for Target population for
API-2 (Baseline 8-11%) analyzed by dose/weight (mg/kg) by Visit

| | Concentration Group | | | | |
|---|---|---|---|---|---|
| | | 1.4-2.7 | | | 5.1-12.4 |
| | | mg/kg | 2.8-4.0 | 4.1-5.0 | mg/kg |
| Visit | Placebo | (<2.7) | mg/kg | mg/kg | (>5.0) |
| Week 4 | −0.14 | −0.37 | −0.47 | −0.36 | −0.26 |
| Week 8 | −0.15 | −0.69 | −0.86 | −0.78 | −0.34 |
| Week 12 | −0.07 | −0.79 | −1.09 | −0.85 | −0.34 |

What is claimed is:

1. A method of treating type 2 diabetes in a human subject comprising administering to a subject in need thereof between 30 to 150 mg daily of a glucagon-like peptide 1 receptor (GLP1R) agonist, wherein the GLP1R agonist is (S)-2-[{(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing, wherein the subject's HbAc1 level is lowered.

2. The method of claim 1, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

3. The method of claim 1, wherein the GLP1R agonist is orally administered.

4. The method of claim 1, wherein the GLP1R agonist is administered daily for at least 3 weeks.

5. The method of claim 1, wherein the GLP1R agonist is administered one time a day or two times a day.

6. The method of claim 5, wherein the GLP1R agonist is administered with food.

7. The method of claim 1, wherein the level of HbA1c in the subject is lowered by at least 0.3 percentage points where the level of HbA1c is measured as a percentage according to the National Glycohemoglobin Standardization Program (NGSP) protocol.

8. The method of claim 7, wherein the level of HbA1c in the subject is lowered by at least 0.5 percentage points.

9. The method of claim 1, wherein from 0.5 to 1.5 mg of the GLP1R agonist per kilogram of the subject's body weight is administered daily.

10. A method of lowering the level of HbA1c in a human subject having type 2 diabetes, the method comprising administering to a subject in need thereof less than 1.35 mg/kg daily of a glucagon-like peptide 1 receptor (GLP1R) agonist, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing.

11. The method of claim 10, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

12. The method of claim 10, wherein the GLP1R agonist is orally administered.

13. The method of claim 10, wherein the GLP1R agonist is administered daily for at least 3 weeks.

14. The method of claim 10, wherein the GLP1R agonist is administered one time a day or two times a day.

15. The method of claim 14, wherein the GLP1R agonist is administered with food.

16. The method of claim 10, wherein the level of HbA1c in the subject is lowered by at least 0.3 percentage points where the HbA1c level is measured as a percentage according to the National Glycohemoglobin Standardization Program (NGSP) protocol.

17. The method of claim 16, wherein the level of HbA1c in the subject is lowered by at least 0.5 percentage points.

18. The method of claim 10, wherein the amount of GLP1R administered daily is between 30 mg and 150 mg.

19. A method of improving glycemic control in a human subject with type 2 diabetes, comprising administering to a subject in need thereof less than 1.35 mg/kg daily of a glucagon-like peptide 1 receptor (GLP1R) agonist, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid, a mass-equivalent of a pharmaceutically acceptable salt thereof, or any combination of the foregoing, wherein the subject's fasting plasma glucose (FPG) is reduced relative to a baseline FPG measurement prior to administering the GLP1R agonist.

20. The method of claim 19, wherein the GLP1R agonist is (S)-2-{[(3S,8S)-3-[4-(3,4-dichloro-benzyloxy)-phenyl]-7-((S)-1-phenyl-propyl)-2,3,6,7,8,9-hexahydro-[1,4]dioxino[2,3-g]isoquinoline-8-carbonyl]-amino}-3-[4-(2,3-dimethyl-pyridin-4-yl)-phenyl]-propionic acid hydrochloride (1:2).

21. The method of claim 19, wherein the GLP1R agonist is orally administered.

22. The method of claim 19, wherein the GLP1R agonist is administered daily for at least 3 weeks.

23. The method of claim 19, wherein the GLP1R agonist is administered one time a day or two times a day.

24. The method of claim 23, wherein the GLP1R agonist is administered with food.

25. The method of claim 19, wherein the subject's FPG baseline measurement is at least 140 mg/dL.

26. The method of claim 25, wherein the subject's FPG level is lowered relative to the subject's FPG baseline measurement by at least 10 mg/dL following daily administration of the GLP1R agonist for a period of 12 weeks.

27. The method of claim 19, wherein the subject's FPG level is lowered relative to baseline by at least 30 mg/dL following daily administration of the GLP1R agonist for a period of 12 weeks.

28. The method of claim 27, wherein the amount of GLP1R agonist administered daily is between 30 to 150 mg.
